(19) United States
(12) Patent Application Publication
(10) Pub. No.: US 2006/0235373 A1
(43) Pub. Date: Oct. 19, 2006

(54) Intraluminal heat treatment of a lumen combined with cooling of tissue proximate the lumen

(75) Inventors: Olav Balle-Petersen, Taastrup (DK); Dennis Bo Hansen, Kobenhavn O (DK); Bjarne Asah, Taastrup (DK)

Correspondence Address: LEE & MORSE, P.C., 3141 FAIRVIEW PARK DRIVE, SUITE 500, FALLS CHURCH, VA, 22042, US

(73) Assignee: Asah Medico A/S

(21) Appl. No.: 11/328,466

(22) Filed: Jan. 10, 2006

Related U.S. Application Data
(60) Provisional application No. 60/671,098, filed on Apr. 14, 2005.

(51) Int. Cl. A61B 18/18

(52) U.S. Cl. 606/15

(57) ABSTRACT

An apparatus for intraluminal energy treatment and method thereof includes an energy source for insertion into a lumen of a body and having an output end for energy-emission within the lumen and a cooling device with a cooling member adapted to be positioned on the skin surface proximate the lumen along a part of the lumen to be treated for cooling tissue proximate that part of the lumen.

### FIELD OF THE INVENTION

The present invention relates generally to a method and an apparatus for intraluminal heat treatment of a lumen, more particularly to a method and an apparatus for intraluminal heat treatment in combination with cooling of tissue proximate the lumen.

### BACKGROUND OF THE INVENTION

A number of ways for treatment of vascular diseases, e.g., thrombosis or varicosis, are available. The least invasive treatments use an energy delivery arrangement for providing energy intraluminally to change the fluid dynamics of a blood vessel.

In U.S. Pat. No. 5,053,033 to Clarke and U.S. Pat. No. 6,398,777 to Navarro et al., laser energy is delivered endoluminally. Spanish Patent No. ES2132028 to Salat et al. describes an endoluminal electro-coagulator for varicose vein operations. U.S. Pat. No. 6,613,045 to Laufer et al. describes endoluminal provision of radio frequency energy, microwave energy, or thermal energy.

In using the above treatments, at least local anesthesia, and sometimes even general anesthesia, is required during intraluminal treatment, e.g., during varicose vein surgery.

### SUMMARY OF THE INVENTION

The present invention is therefore directed to a method and apparatus for intraluminal heat treatment, which substantially overcome one or more of the problems due to the limitations and disadvantages of the related art

It is a feature of an embodiment of the present invention to provide a method and an apparatus for intraluminal heat treatment of a lumen in combination with cooling of tissue proximate the lumen during intraluminal treatment so that the need for anesthesia is eliminated.

It is another feature of an embodiment of the present invention to provide a method and an apparatus for cooling the skin of a patient during intraluminal heat treatment, significantly reducing or eliminating influence of the treatment on tissue adjacent the target tissue subjected to the heat treatment.

It is yet another feature of an embodiment of the present invention to monitor a positioning of the apparatus for intralumninal heat treatment.

It still another feature to control a temperature of a cooling medium delivered to the skin of a patient during intralumninal heat treatment.

At least one of the above and other features and advantages of the present invention may be realized by providing a method for intraluminal heat treatment of a patient, including locating a part of a lumen to be treated, positioning a cooling member on the skin surface proximate the lumen along a part of the lumen to be treated for cooling tissue proximate that part of the lumen, inserting an energy-emitting member into the lumen, and moving the energy-emitting member along the longitudinal extension of the part of the lumen to be treated for heating of the lumen wall by energy emission. Locating may be by visual inspection of the appearance of the patient's body.

At least one of the above and other features and advantages of the present invention may be realized by providing an apparatus for intraluminal energy treatment, including an energy source for insertion into a lumen of a body and having an output end for energy-emission within the lumen, and a cooling device with a cooling member adapted to be positioned on the skin surface proximate the lumen along a part of the lumen to be treated for cooling tissue proximate that part of the lumen.

The cooling member may be a flexible bag with channels for the flow of cooling medium. The bag may be made of plastic, e.g., soft PVC. Further, the bag may be divided into a meander shaped flow channel by internal walls forming a flow path for the cooling medium from the input to the output. The flexibility of the bag enables it to substantially fit and contact the skin surface of the patient's body proximate the lumen to be treated without compressing the tissue underneath the bag. Hereby, an improved cooling of the entire surface is obtained and it is possible to apply the cooling member without compressing the lumen.

In another embodiment, the cooling device may include an opening, such as a spray nozzle, for emission of a cooling spray, e.g., an air flow, a cryogenic spray, etc., onto the skin proximate to the output end.

In yet another embodiment, the cooling device may include an opening, such as a spray nozzle, for emission of a cooling spray, such as an air flow, a cryogenic spray, etc., onto a cooling member having a contact surface adapted to be in contact with the skin during treatment.

The cooling member may further include a window for transmission of energy emitted by the output end facilitating detection of the position of the output end when the cooling device is positioned at the skin proximate the output end. The window may be transparent to visible light, e.g., light having a wavelength between about 600 nm to 700 nm, or about 500 nm to 550 nm, so that the operator may observe light penetrating the skin beneath the cooling member during treatment. The window may constitute the contact surface or may form a part of the contact surface.

The window may form an upper and a lower wall of the compartment, the lower wall constituting the contact surface being in contact with skin during treatment and the upper wall facing away from the skin during treatment so that energy may be transmitted through the walls and the cooling medium in the compartment during treatment facilitating observation of the treatment area.

In another embodiment, the window may be a cooling member in contact with the compartment for transmission of heat from the skin to the cooling medium and allowing energy to be transmitted through the window during treatment facilitating observation of the treatment area.

The window may be provided with a distance indicator permitting an operator of the apparatus to observe the distance of retraction or insertion of the fiber output end within the lumen by observation of visible light emitted from the output end and transmitted through the lumen wall and tissue proximate the lumen to the skin surface.

The member contact surface may have a recessed section whereby the lumen may remain fully or partially uncompressed when the contact surface is pressed against the skin surface proximate the lumen.

It is an advantage of the present invention that the need for local anesthesia of treatment sites is significantly reduced or eliminated.

It is another advantage of the present invention that influence of the treatment on tissue adjacent target tissue to be treated by the emitted energy is significantly reduced or eliminated.

### BRIEF DESCRIPTION OF THE DRAWINGS

The above and other features and advantages of the present invention will become more apparent to those of ordinary skill in the art by describing in detail exemplary embodiments thereof with reference to the attached drawings in which:

FIG. 1 illustrates a schematic view of a cooling member of an apparatus according to a first embodiment of the present invention,

FIG. 2 illustrates a schematic view of an apparatus according to a second embodiment of the present invention for spraying a cooling medium onto the skin at the treatment site,

FIG. 3 illustrates a schematic view of an apparatus according to a third embodiment of the present invention for spraying a cooling medium onto a cooling member,

FIG. 4 illustrates a schematic view of a cooling member of an apparatus according to a fourth embodiment of the present invention having a recess for reducing compression of the lumen to be treated,

FIG. 5 illustrates a perspective view of another cooling device in accordance with a fifth embodiment of the present invention, and

FIG. 6 illustrates a perspective view of a cooling device including a cooling member having a flexible bag in accordance with a sixth embodiment of the present invention.

### DETAILED DESCRIPTION OF THE INVENTION

U.S. Provisional Patent Application No. 60/671,098, filed on Apr. 14, 2005, in the U.S. Patent & Trademark Office, and entitled: “Skin Cooling Apparatus,” is incorporated by reference herein in its entirety for all purposes.

The present invention will now be described more fully hereinafter with reference to the accompanying drawings, in which exemplary embodiments of the invention are shown. The invention may, however, be embodied in different forms and should not be construed as limited to the embodiments set forth herein. Rather, these embodiments are provided so that this disclosure will be thorough and complete, and will fully convey the scope of the invention to those skilled in the art. In the figures, the dimensions of layers and regions are exaggerated for clarity of illustration. Like reference numerals refer to like elements throughout.

Throughout this document the terms “intraluminal treatment” or “endoluminal treatment” are to mean a treatment during which energy is emitted within a lumen of a patient for treatment of tissue forming a lumen wall or treatment of tissue resident in or proximate to the lumen.

For example, varicose veins may be treated by laser light. During treatment an optical fiber coupled to a laser is introduced into the vein lumen to deliver intraluminal laser energy within the lumen from the output end of the fiber.

For example, the fiber may be operatively coupled to a laser emitting energy in the wavelength range from 500 nm to 1800 nm. Energy in this wavelength range is effectively absorbed by hemoglobin in the blood whereby the blood is heated and the heated blood in turn heats the vein wall. Further, in this wavelength range, human fatty tissue has lower energy absorption than collagen so that energy in this wavelength range passing the vein wall will do little damage to the tissue surrounding the vein.

Heating of the vein wall causes fibrosis of the vein wall leading to a decrease in the diameter of the vein and removal of the vein during the subsequent healing. The amount of fibrosis in the vein wall is determined by the amount of laser energy delivered to the wall. Preferably, the vein wall is damaged to an extent that the subsequent fibrosis causes the vein to collapse.

The optical fiber may be inserted through an angiocatheter placed percutaneously into the vein to be treated.

In another example, thrombosis is treated by removal of material in the lumen thereby enlarging the lumen.

Thus, the energy source may include a light source for emission of treatment light, such as a laser, such as a laser having a wavelength ranging from 800 nm to 820 nm, or from 930 nm to 950 nm, or from 970 nm to 990 nm, a 810 nm diode laser, a 940 nm diode laser, a 980 nm diode laser, etc., and an optical fiber coupled to the laser source for insertion into the lumen and having an output end for emission of the treatment light. The power of light output by the laser light may be between about 5 W to 20 W, e.g., from about 8 W to 15 W. The light may be delivered in pulses having a duration of less than about 2 seconds, e.g., between 0.5 to 1.5 seconds.

The energy may be emitted in pulses as controlled by the operator of the apparatus, e.g., actuation of a foot pedal. Upon pulse emission, the fiber end may be moved from about mm to 10 mm, preferably about 3 mm to 4 mm, along the lumen before a new pulse emission. At the start of energy emission, dissipated heat may cause expansion of the lumen whereby the detected energy level at the skin increases. This may be utilized for recording of the amount of energy delivered within the lumen during the entire treatment.

Alternatively, the laser light may be emitted continuously. For such continuous illumination, the fiber may be moved at an appropriate speed, e.g., about 5 mm per second.

Alternatively, the energy source may include a radio frequency source, such as a bipolar generator, and a set of electrodes coupled to the generator for insertion into the lumen and having an output end for creating a heated region of the lumen wall. Alternatively, a microwave source or a heat source may be used.

Energy emitted within the lumen may inadvertently pass the lumen wall and be absorbed by tissue surrounding the lumen thereby heating the tissue proximate the lumen. In order to keep the temperature of tissue proximate the treated lumen sufficiently low to avoid damaging this tissue, the tissue may be cooled by a cooling member positioned on the skin surface proximate the lumen along a part of the lumen to be treated. Preferably, cooling is performed before, during and after emission of energy within the lumen so that a damaging temperature increase of tissue proximate the treated lumen is effectively avoided. However, cooling may be applied before the energy is emitted, simultaneous with the energy being emitted, after the energy has been emitted, or in any combination hereof as appropriate.

Preferably, the cooling member has a size sufficiently large to cool tissue proximate the lumen without being moved during the treatment whereby effective cooling of influenced tissue is ensured during the entire treatment. Further, movement of the cooling member adds waiting time to the treatment until the tissue at the new position of the member has been cooled sufficiently for the treatment to continue. Such waiting time is avoided with a sufficiently large cooling member.

The contact surface of the cooling member may have a width of at least about 1 cm, preferably at least about 3 cm, more preferably at least about 5 cm, and a length of at least about 5 cm, preferably least about 6 cm, more preferably at least about 10 cm. A rectangular area of this size permits cooling to be applied to an appropriate area proximate the treated lumen. The cooling member may have an area sufficient for substantially covering a treatment area, e.g., the front surface of the thigh of a patient.

The cooling member may be a flexible cooling member capable of comfortably fitting onto a curved surface of a human.

The cooling member may have a compartment for accommodation of a cooling medium. The cooling member may further have an input and an output for cooling medium interconnecting the compartment and a refrigerator for recirculation and cooling of the cooling medium. The cooling medium may be water or a water/alcohol mixture, and may be refrigerated. The temperature of the cooling medium may be controlled in such a way that the contact surface of the cooling member has a temperature ranging from about −1° C. to +15° C., preferably from about −1° C. to +10° C.

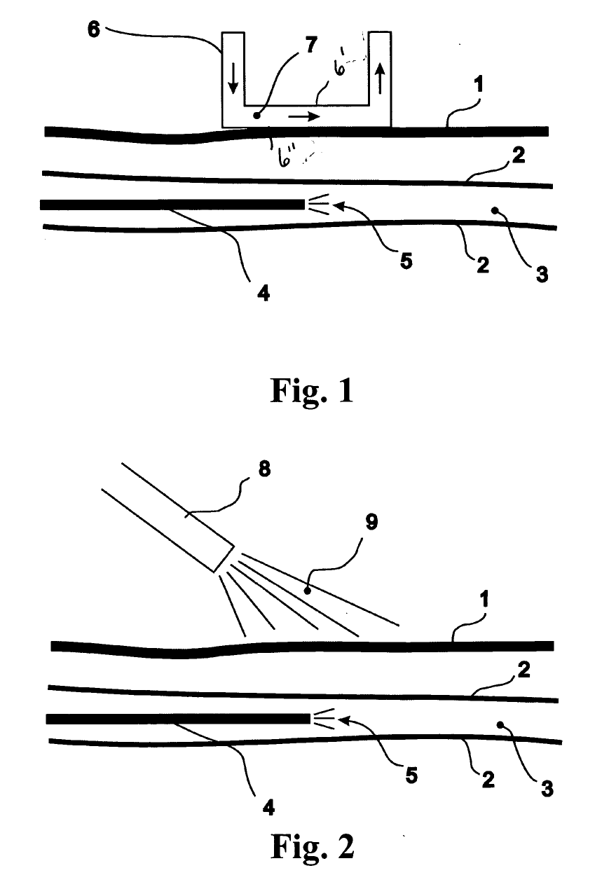

Details of a cooling device in accordance with the embodiments of the present invention are described in detail below, with reference to FIGS. 1-6.

FIG. 1 illustrates a schematic view of a first embodiment of the present invention. The apparatus may include a laser (not shown) coupled to an optical fiber 4 for emission of light 5 at its output end. The optical fiber may be positioned inside the lumen 3 of the vein 2, e.g., the greater saphenous vein, to be treated. A cooling member 6 may be positioned in contact with the skin 1 of the patient for cooling the skin proximate the output end and the intervening tissue. The cooling member may include a compartment for accommodation of a cooling medium 7. The cooling compartment may be connected to cooling channels (not shown), which in turn are connected to a refrigerator (not shown) for recirculation and cooling of the cooling medium. The cooling medium may be a gas, such as a non ozone-depleting gas, or a liquid, such as a water/alcohol mixture, etc. The temperature of the cooling medium may be controlled in such a way that a contact surface of the cooling member 6 has a temperature ranging from about −1° C. to +10° C.

The cooling member 6 may include a window for transmission of light emitted by the output end facilitating detection of the position of the output end when the cooling member 6 is positioned at the skin 1 proximate the output end so that the operator may observe the skin beneath the cooling member 6 during treatment. The window may form an upper wall 6′ and a lower wall 6″ of the compartment, the lower wall 6″ serving as the contact surface to be in contact with skin during treatment and the upper wall 6′ facing away from the skin during treatment so that light is transmitted through the walls and the cooling medium 7 in the compartment during treatment facilitating observation of the treatment area. In the event that the treatment light is not visible, a further light source (not shown) may be coupled to the optical fiber 4 for additional emission of visible light from the output end for transmission through the window and the cooling medium 7 facilitating observation of the position of the output end within the lumen in relation to the skin.

By emission of visible light, the visible output light can be seen through the skin and may be emitted simultaneously with light of another wavelength. Thus, the apparatus according to the invention may further include a light source for emission of visible light that is coupled to the optical fiber whereby the visible light is output from the output end of the optical fiber. The visible light emitted by the output end may be used for detection through the skin of the position of the output end. An operator of the apparatus may determine the position by observing and locating the visible spot on the skin of the patient to be treated. The visible spot may indicate the skin position closest to the output end within the lumen.

The window may include ruler like distance indications, e.g., in the form of at least two marks on the window having a certain distance. Such distance indications permit the operator to control the distance of retraction or insertion of the fiber output end within the lumen by observation of visible light emitted from the output end and transmitted through the lumen wall and tissue proximate the lumen to the skin surface.

FIG. 2 illustrates a schematic view of a second embodiment of the present invention. As shown in FIG. 2, a cooling device may include a spray nozzle 8 for emission of a cooling medium 9 onto the skin proximate the endoluminal treatment site, i.e., the position of the output end, for cooling of the skin proximate the output end and the intervening tissue. The cooling medium 9 may be, e.g., cold air, cold gas, cold vapor, or cryogenic liquid drops. The cooling medium 9 may be added in a sequence of bursts or as a steady stream to maintain the desired temperature of the skin. The temperature of the cooling medium 9 may be controlled so that the skin of the patient is cooled to a temperature between about −1° C. and +10° C.

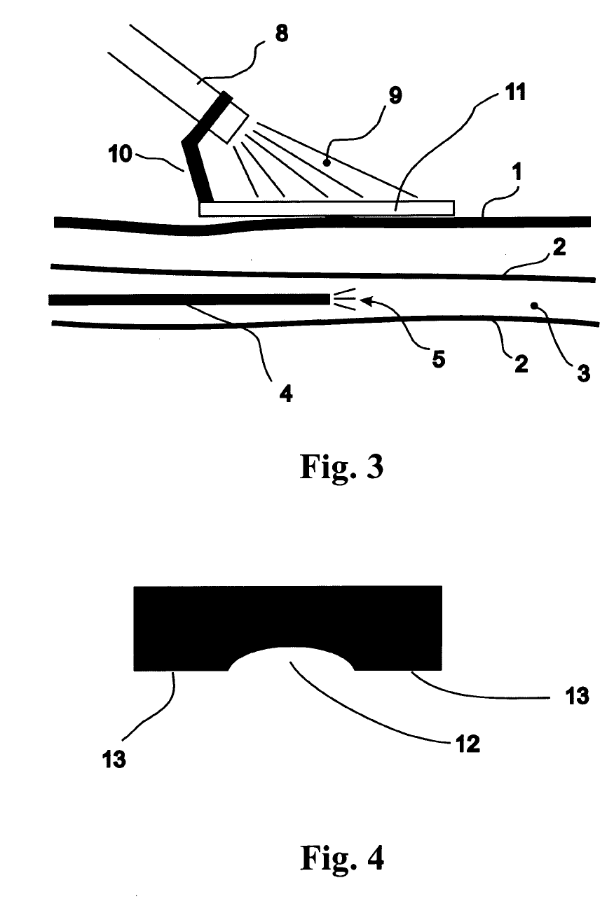

FIG. 3 illustrates a schematic view of a third embodiment of the present invention, which the same as the second embodiment shown in FIG. 2, except further includes a cooling member 11 positioned in contact with the skin of the patient. The spray nozzle 8 emits the cooling medium 9 onto the cooling member 11, thereby indirectly cooling the skin. The cooling medium 9 may be cold air, cold gas, cold vapor, or cryogenic liquid drops. The temperature of the cooling medium 9 may be controlled so that the skin of the patient is cooled to a temperature between −1° C. and +10° C.

FIG. 4 illustrates a schematic view of a cooling member 13 of an apparatus according to a third embodiment of the present invention. The cooling member 13 may include a recess 12 for reducing compression of the lumen to be treated. For example, during treatment of a blood vessel, the recessed section 12 may prevent pressing blood out of the vessel, hence the blood is allowed to stay at the treatment site and absorb and conduct the energy 5 emitted in the lumen 3 of the blood vessel 2. Preferably, the recessed section extends from one edge of the cooling member 13 to the opposite edge and has a width of between 5 mm and 10 mm, and a depth of between 5 mm and 10 mm. The cooling members shown in FIGS. 1 and 3 may include such a recess 12.

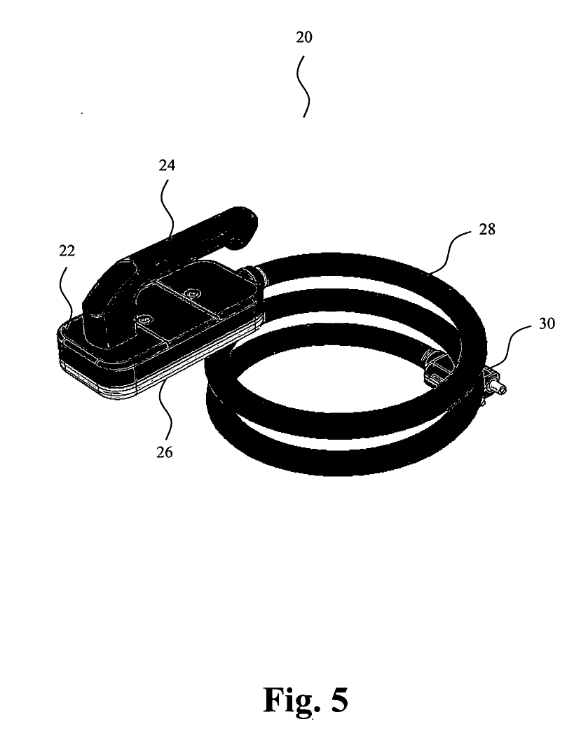

FIG. 5 illustrates a perspective view of a cooling device 20 according to a fifth embodiment of the present invention. The cooling device 20 may include a cooling member 22 with a handle 24 for manual positioning of the cooling member 22 on the skin surface of a patient proximate the lumen to be treated. The cooling member 22 may have a cooling surface 26 to be positioned in contact with the skin of the patient for cooling the skin, .e.g., above the veins to be treated and the intervening tissue. The cooling member 20 may include a compartment (not visible) for accommodation of a cooling medium. The cooling compartment may be connected to input and output cooling channels residing in the hose 28, which in turn are connected with connector 30 to a refrigerator (not shown) for recirculation and cooling of the cooling medium. The cooling medium may be a water/alcohol mixture. The temperature of the cooling medium may be controlled such that the contact surface 26 of the cooling member 22 has a temperature ranging from −1° C. to +10° C. The cooling member 22 may be of sufficient size such that an entire surface of a treatment area, e.g., a front surface of the patient's thigh, may be cooled without a requiring movement of the member 22 during treatment so that the treatment is not delayed by such movement that would include waiting time until the tissue at the new position of the member 22 has been cooled sufficiently for the treatment to continue.

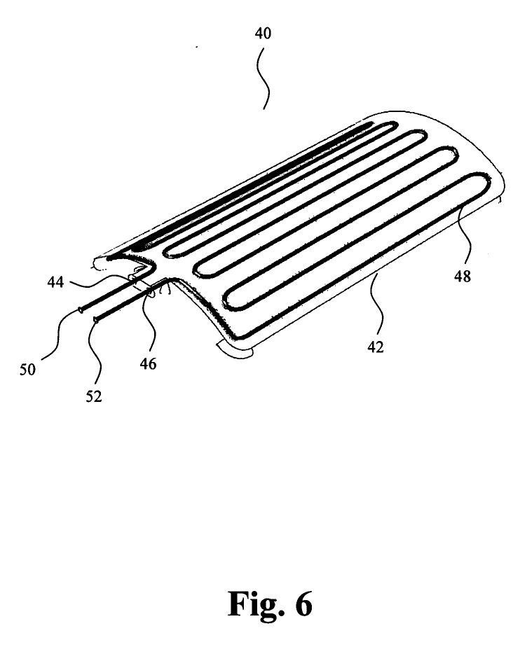

FIG. 6 illustrates a perspective view according to a sixth embodiment of the present invention, in which a cooling device 40 includes a flexible bag 42 as a cooling member. The bag 42 may have an input 44 and an output 46 for cooling medium and internal walls dividing the internal volume of the bag 42 into a meander shaped flow channel 48 forming a flow path for the cooling medium from the input 44 to the output 46. The input 42 and the output 46 may be connected through respective hoses 50, 52 to a refrigerator (not shown) for recirculation and cooling of the cooling medium. The cooling medium may be water or a water/alcohol mixture. The temperature of the cooling medium may be controlled such that the contact surface of the bag 42 has a temperature ranging from about −1° C. to +10° C. The bag 42 may be of sufficient size so that an entire surface of the treatment area, e.g., the front surface of the patient's thigh, may be cooled without a requiring movement of the bag 42 during treatment. Thus, treatment delays may be avoided, since movement would include waiting time until tissue at the new position of the bag 42 has been cooled sufficiently for the treatment to continue.

The material of the bag may be transparent and may be flexible. The bag 42 may be made of plastic, e.g., soft PVC. The flexibility of the bag 42 enables it to substantially fit the contour of the patient's skin surface, e.g., the skin surface of a thigh along substantially the entire front surface of the thigh, without necessarily compressing the tissue underneath the bag 42.

As can be seen from the above embodiment, the window may be made from a material having a crystalline structure, such as sapphire or quartz, e.g., a synthetic sapphire. The thermal conductivity of the window may be at least about 25 W/m° C. The cooling medium may be thermally coupled to the window along the edges of the window, it may flow through channels in the window, or it may flow over the surface of the window.

Different features of the different embodiments may be used in conjunction with each other. Further, as noted in the discussion of the first embodiment, the position of the output end of the optical fiber may be monitored by tracking visible light output from the output end. Such monitoring may also be realized using ultrasonic imaging or Doppler ultrasound imaging, to visualize the position of the fiber output end, e.g., during insertion of the fiber into the lumen, such as a vein, and before and after movement of the fiber in the lumen during treatment to verify correct positioning of the output end, on a display unit, e.g., a CRT screen, a LCD screen, etc. Further, the lumen and the lumen walls may be shown on the display unit, e.g., to monitor changes of lumen shape or dimensions before and after emission of treatment light to verify the effect of the treatment.

The apparatus may also include a detector, such as a photo detector, etc, for detection of emitted visible light whereby the amount of emitted treatment energy may be recorded. At the start of energy emission, dissipated heat may cause expansion of the lumen whereby the level of visible light at the skin increases. This may be utilized for recording of the number of energy pulses delivered within the lumen and thereby recording of the total amount of energy delivered during the entire treatment. The detector may further be adapted for sensing change in the intensity of a substantially monochromatic radiation. This information may be fed back to control the lights source, e.g., the laser.

Exemplary embodiments of the present invention have been disclosed herein, and although specific terms are employed, they are used and are to be interpreted in a generic and descriptive sense only and not for purpose of limitation. Accordingly, it will be understood by those of ordinary skill in the art that various changes in form and details may be made without departing from the spirit and scope of the present invention as set forth in the following claims.

What is claimed is:

1. A method for intraluminal energy treatment of a patient, comprising:
locating a part of a lumen to be treated;
positioning a cooling member on the skin surface proximate the lumen along a part of the lumen to be treated for cooling tissue proximate the part of the lumen;
inserting an energy-emitting member into the lumen; and
moving the energy-emitting member along the longitudinal extension of the part of the lumen to be treated for heating of the lumen wall by energy emission.

2. The method for intraluminal energy treatment as claimed in claim 1, wherein the cooling member is a flexible cooling member.

3. The method for intraluminal energy treatment as claimed in claim 1, wherein the energy-emitting member is an optical fiber with an output end for emission of light into the lumen.

4. The method for intraluminal energy treatment as claimed in claim 2, wherein the energy-emitting member is an optical fiber with an output end for emission of light into the lumen.

5. The method for intraluminal energy treatment as claimed in claim 1, wherein the internal volume of a vein constitutes the lumen.

6. The method for intraluminal energy treatment as claimed in claim 2, wherein the internal volume of a vein constitutes the lumen.

7. The method for intraluminal energy treatment as claimed in claim 3, wherein the internal volume of a vein constitutes the lumen.

8. The method for intraluminal energy treatment as claimed in claim 4, wherein the internal volume of a vein constitutes the lumen.

9. An apparatus for intraluminal energy treatment, comprising:
an energy source for insertion into a lumen of a body and having an output end for energy-emission within the lumen; and
a cooling device with a cooling member adapted to be positioned on the skin surface proximate the lumen along a part of the lumen to be treated for cooling tissue proximate that part of the lumen.

10. The apparatus as claimed in claim 9, wherein the cooling member has a recess to be positioned proximate the lumen to be treated thereby substantially avoiding compression of the lumen during treatment.

11. The apparatus as claimed in claim 9, wherein the cooling member has a cooling surface that is adapted to substantially cover the front surface of a human thigh.

12. The apparatus as claimed in claim 9, wherein the cooling device comprises a flexible cooling member that is adapted for comfortably fitting onto a curved surface of a human.

13. The apparatus as claimed in claim 11, wherein the cooling device comprises a flexible cooling member that is adapted for comfortably fitting onto a curved surface of a human.

14. The apparatus as claimed in claim 12, wherein the flexible cooling member has an input and an output for cooling fluid and internal walls dividing the internal volume of the cooling member into a meander shaped flow channel forming a flow path for the cooling fluid from the input to the output.
